(19) United States
(12) Patent Application Publication
(10) Pub. No.: US 2005/0014766 A1
(43) Pub. Date: Jan. 20, 2005

(54) Use of n-(indolecarbonyl-)piperazine derivatives

(76) Inventors: Christoph van Amsterdam, Darmstadt (DE); Ewen Sedman, Hampshire (DE); Gerd Bartoszyk, Weiterstadt (DE); Jurgen Hellmann, Ober-Ramstadt (DE); Friedrich von Landerberg, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/496,572

(22) PCT Filed: Oct. 28, 2002
(86) PCT No.: PCT/EP02/12009

(51) Int. Cl. A61K 31/496

(52) U.S. Cl. 514/254.09; 514/253.09

(57) ABSTRACT

Use of compounds of the formula (I) in which R¹, R⁴, R², R⁴ and R⁵ are as defined in claim 1, and physiologically acceptable salts and solvates thereof, for the treatment of obesity, sub-types of anxiety, sub-types of schizophrenia and types of dementia of various origin.

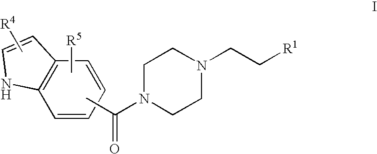

The invention relates to the use of compounds of the formula I 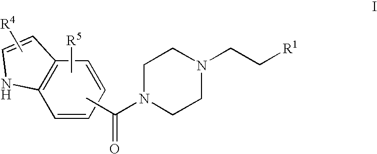
in which
R¹ is a phenyl radical or naphthyl radical, each of which is unsubstituted or substituted by R² and/or R³, or is Het¹,
R² and R³ are each, independently of one another, Hal, A, OA, OH or CN,
R⁴ and R⁵ are each, independently of one another, H, CN, acyl, Hal, A, OA, OH, CONH₂, CONHA or CONA₂,
R⁴ and R⁵ together are alternatively alkylene having 3-5 carbon atoms,
Het¹ is a monocyclic or bicyclic, unsaturated heterocyclic ring system which is unsubstituted or monosubstituted or disubstituted by Hal, A, OA or OH and which contains one, two or three identical or different heteroatoms, such as nitrogen, oxygen and sulfur,
A is alkyl having 1-6 carbon atoms,
Hal is F, Cl, Br or I,
   and in which the indole ring may also be replaced by an isatin unit, and physiologically acceptable salts and solvates thereof, for the preparation of a medicament for the treatment of obesity, sub-types of anxiety, sub-types of schizophrenia and types of dementia of various origin.

For II radicals which occur more than once, such as, for example, A or Hal, their meanings are independent of one another.

The radical A is alkyl and has from 1 to 6, preferably 1, 2, 3 or 4, in particular 1 or 2, carbon atoms. Alkyl is therefore in particular, for example, methyl, furthermore ethyl, n-propyl, isopropyl, n-butyl, sec-butyl or tertbutyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl.

Acyl preferably has 1-6 carbon atoms and is, for example, formyl, acetyl, propionyl, butyryl, furthermore trifluoroacetyl.

Alkylene is propylene, butylene or pentylene.

OA is preferably methoxy, furthermore also ethoxy, n-propoxy, isopropoxy, n-butoxy, isobutoxy, sec-butoxy or tert-butoxy.

Hal is fluorine, chlorine, bromine or iodine, in particular fluorine or chlorine.

R¹ is phenyl or naphthyl, each of which is unsubstituted or preferably monosubstituted—as indicated—specifically preferably phenyl, o-, m- or ptolyl, o-, m- or p-ethylphenyl, o-, m- or p-propylphenyl, o-, m- or p-isopropylphenyl, o-, m- or p-tert-butylphenyl, o-, m- or p-trifluoromethylphenyl, o-, m- or p-hydroxyphenyl, o-, m- or p-nitrophenyl, o-, m- or p-(trifluoromethoxy)phenyl, o-, m- or p-cyanophenyl, o-, m- or p-methoxyphenyl, o-, m- or p-ethoxyphenyl, o-, m- or p-fluorophenyl, o-, m- or p-bromophenyl, o-, m- or p-chlorophenyl, o-, m- or p-(difluoromethoxy)phenyl, o-, m- or p-(fluoromethoxy)phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2-chloro-3-methyl-, 2-chloro-4-methyl-, 2-chloro-5-methyl-, 2-chloro-6-methyl-, 2-methyl-3-chloro-, 2-methyl-4-chloro-, 2-methyl-5-chloro-, 2-methyl-6-chloro-, 3-chloro-4-methyl-, 3-chloro-5-methyl- or 3-methyl-4-chlorophenyl, 2-bromo-3-methyl-, 2-bromo-4-methyl-, 2-bromo-5-methyl-, 2-bromo-6-methyl-, 2-methyl-3 -bromo-, 2-methyl-4-bromo-, 2-methyl-5-bromo-, 2-methyl-6-bromo-, 3-bromo-4-methyl-, 3-bromo-5-methyl- or 3-methyl-4-bromophenyl, 2,4- or 2,5-dinitrophenyl, 2,5- or 3,4-dimethoxyphenyl, 3-nitro-4-chlorophenyl, 2,3,4-, 2,3,5-, 2,3,6-, 2,4,6- or 3,4,5-trichlorophenyl, 2,4,6-tri-tert-butylphenyl, furthermore preferably 2-nitro-4-(trifluoromethyl)phenyl, 3,5-di(trifluoromethyl)phenyl, 2,5-dimethylphenyl, 2-hydroxy-3,5-dichlorophenyl, 2-fluoro-5- or 4-fluoro-3-(trifluoromethyl)phenyl, 4-chloro-2- or 4-chloro-3-(trifluoromethyl)-, 2-chloro-4- or 2-chloro-5-(trifluoromethyl)phenyl, 4-bromo-2- or 4-bromo-3-(trifluoromethyl)phenyl, p-iodophenyl, 2-nitro-4-methoxyphenyl, 2,5-dimethoxy-4-nitrophenyl, 2-methyl-5-nitrophenyl, 2,4-dimethyl-3-nitrophenyl, 4-fluoro-3-chlorophenyl, 4-fluoro-3,5-dimethylphenyl, 2-fluoro-4-bromophenyl, 2,5-difluoro-4-bromophenyl, 2,4-dichloro-5-methylphenyl, 3-bromo-6-methoxyphenyl, 3-chloro-6-methoxyphenyl, 2-methoxy-5-methylphenyl or 2,4,6-triisopropylphenyl.
R¹ is also Het¹.
Het¹ is preferably 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or -5-thiazolyl, 3-, 4- or 5-isothiazolyl, 2-, 3- or 4-pyridyl, 2-, 4-, 5- or 6-pyrimidinyl, furthermore preferably 1,2,3-triazol-1-,-4- or-5-yl, 1,2,4-triazol-1-,-3- or 5-yl, 1- or 5-tetrazolyl, 1,2,3-oxadiazol-4-or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 2-, 3-, 4-, 5- or 6-2H-thiopyranyl, 2-, 3- or 4-4-H-thiopyranyl, 3- or 4-pyridazinyl, pyrazinyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl.
R¹ is very particularly preferably phenyl, p-chlorophenyl, p-fluorophenyl, thiophen-2-yl, 5-chlorothiophen-2-yl, 2,5-dichlorothiophen-3-yl or 2- or 3-furyl.
R⁴ and R⁵ are each, independently of one another, preferably H, Hal, alkyl having 1-6 carbon atoms, alkoxy having 1-6 carbon atoms or hydroxyl, furthermore cyano or acyl.
R⁴ is preferably H, Hal, A, OA, OH, CN, acyl, CONH₂ or CONHA. R⁵ is preferably H.

Preference is given to the compounds of the formula I in which the R¹—CH₂—CH₂-piperazinecarbonyl radical substitutes the 4-, 5-, 6- or 7-position of the indole ring.

Accordingly, the invention relates in particular to the use of the compounds of the formula I in which at least one of the said radicals has one of the preferred meanings indicated above. Some preferred groups of compounds can be expressed by the following sub-formulae Ia to Ih, which conform to the formula I and in which the radicals not designated in greater detail are as defined for the formula 1, but in which
in Ia R¹ is phenyl;
in Ib R¹ is phenyl, which is unsubstituted or monosubstituted by Hal;
in Ic R¹ is phenyl or Het¹, each of which is monosubstituted by Hal;
in Id R¹ is phenyl or Het¹, each of which is unsubstituted or monosubstituted by Hal;
in Ie R¹ is phenyl or Het¹, each of which is unsubstituted or monosubstituted by Hal,
   Het¹ is an unsaturated heterocyclic ring system, which is unsubstituted or monosubstituted or disubstituted by Hal or A and which contains one or two identical or different heteroatoms, such as nitrogen, oxygen and sulfur;
in If R¹ is phenyl or Het¹, each of which is unsubstituted or monosubstituted by Hal,
   R⁴ and R⁵ are each, independently of one another, H, Hal or A,
   Het¹ is an unsaturated heterocyclic ring system, which is unsubstituted or monosubstituted or disubstituted by Hal or A and which contains one or two identical or different heteroatoms, such as nitrogen, oxygen and sulfur;
in Ig R¹ is phenyl or Het¹, each of which is unsubstituted or monosubstituted by Hal,
   R⁴ and R⁵ are each, independently of one another, H, Hal or A,
   R⁴ and R⁵ together are alternatively alkylene having 3-5 carbon atoms,
   Het¹ is thienyl or furyl, each of which is unsubstituted or monosubstituted or disubstituted by Hal or A;
in Ih R¹ is phenyl or Het¹, each of which is unsubstituted or monosubstituted by Hal,
   R⁴ are each, independently of one another, H, Hal, CN, acyl or A,
   R⁵ is H,
   R⁴ and R⁵ together are alternatively alkylene having 3-5 carbon atoms,
   Het¹ is thienyl or furyl, each of which is unsubstituted or monosubstituted or disubstituted by Hal or A.
in Ii R¹ is phenyl, naphthyl or Het¹, each of which is unsubstituted or monosubstituted by Hal,
   R⁴ are each, independently of one another, H, Hal, CN, acyl, A or CONH₂,
   R⁵ is H,
   R⁴ and R⁵ together are alternatively alkylene having 3-5 carbon atoms,
   Het¹ is thienyl or furyl, each of which is unsubstituted or monosubstituted or disubstituted by Hal or A,
      and in which the indole ring may also be replaced by an satin unit.

Use is preferably made in accordance with the invention of the following compounds, which are characterised in greater detail in WO 01/07435—w where appropriate in the form of one of their salts:
(1H-indol-4-yl)(4-phenethylpiperazin-1-yl)methanone,
(1H-indol-4-yl)[4-(4-fluorophenethyl)piperazin-1-yl]methanone,
(1H-indol-4-yl)[4-(2,5-dichlorothiophen-3-ylethyl)piperazin-1-yl]methanone,
(3-formyl-1H-indol-5-yl)[4-(4-fluorophenethyl)piperazin-1-yl]methanone,
(1H-indol-6-yl)[4-(4-fluorophenethyl)piperazin-1-yl]methanone,
(1H-indol-6-yl)[4-(thiophen-2-ylethyl)piperazin-1-yl]methanone, hydrochloride,
(1H-indol-6-yl)[4-(2,5-dichlorothiophen-3-ylethyl)piperazin-1-yl]methanone,
(3-cyano-1H-indol-6-yl)[4-(4-fluorophenethyl)piperazin-1-yl]methanone,
(1H-indol-7-yl)(4-phenethylpiperazin-1-yl)methanone,
(1H-indol-7-yl)[4-(4-fluorophenethyl)piperazin-1-yl]methanone,
(1H-indol-7-yl)[4-(5-chlorothiophen-2-ylethyl)piperazin-1-yl]methanone,
(3-formyl-1H-indol-7-yl)[4-(4-fluorophenethyl)piperazin-1-yl]methanone,
(3-cyano-1H-indol-7-yl)[4-(4-fluorophenethyl)piperazin-1-yl]methanone,
(2,3-dimethyl-1H-indol-7-yl)[4-(4-fluorophenethyl)piperazin-1-yl]methanone,
(6,7,8,9-tetrahydro-5H-carbazol-3-yl)(4-phenethylpiperazin-1-yl)methanone,
(3-formyl-1H-indol-6-yl)[4-(4-fluorophenethyl)piperazin-1-yl]methanone,
(1H-indol-6-yl)[4-(5-chlorothiophen-2-ylethyl)piperazin-1-yl]methanone,
(1H-indol-4-yl)[4-(5-chlorothiophen-2-ylethyl)piperazin-1-yl]methanone,
(3-cyano-1H-indol-5-yl)[4-(4-fluorophenethyl)piperazin-1-yl]methanone,
(3-cyano-1H-indol-7-yl)[4-(naphth-2-ylethyl)piperazin-1-yl]methanone,
(3-cyano-1H-indol-4-yl)[4-(4-fluorophenethyl)piperazin-1-yl]methanone,
(3-cyano-1H-indol-4-yl)[4-(2-fluorophenethyl)piperazin-1-yl]methanone,
(3-cyano-1H-indol-7-yl)[4-(2-fluorophenethyl)piperazin-1-yl]methanone,
(3-aminocarbonyl-1H-indol-7-yl)[4-(4-fluorophenethyl)piperazin-1-yl]-methanone,
(3-cyano-1H-indol-7-yl)[4-(4-fluorophenethyl)piperazin-1-yl]methanone,
(3-cyano-1H-indol-7-yl)[4-(5-chlorothiophen-2-ylethyl)piperazin-1-yl]methanone, (3-cyano-1H-indol-7-yl)(4-phenethylpiperazin-1-yl)methanone,
(3-cyano-1H-indol-7-yl)[4-(2,4-difluorophenethyl)piperazin-1-yl]methanone.

Particular preference is given in accordance with the invention to the use of (3-cyano-1H-indol-7-yl)[4-(4-fluorophenethyl)piperazin-1-yl]methanone and (3-aminocarbonyl-1H-indol-7-yl)[4-(4-fluorophenethyl)piperazin-1-yl]-methanone.

Very particular preference is given to (3-cyano-1H-indol-7-yl)[4-(4-fluorophenethyl)piperazin-1-yl]methanone.

The invention furthermore relates to the use of compounds of the formula I as defined above for the preparation of a medicament for the treatment of sub-types of anxiety states selected from a group consisting of social phobia, specific phobias, neophobia, post-traumatic stress disorders, acute stress anxiety, generalised anxiety disorders and bipolar disorders (mania).

A further aspect of the invention is the use of compounds of the formula I for the preparation of a medicament for the treatment of sub-types of schizophrenia selected from a group consisting of schizotypical personality disorders, prevention of schizophrenia in first-degree relatives, treatment-resistant schizophrenia and psychosis in tardive dyskinesia.

The invention furthermore relates to the use of compounds of the formula I for the preparation of a medicament for the treatment of diseases selected from a group consisting of aggression (including aggression disorders in youths and adults) and behavioural disorders in dementia (in particular in the elderly).

The invention likewise relates to the use of compounds of the formula I for the preparation of a medicament for supplementary treatment in low-dose neuroleptic treatment.

A further aspect of the invention is the use of compounds of the formula I for the preparation of a medicament for the treatment of diseases selected from a group consisting of Parkinson's disease (including idiopathic Parkinson's disease) and attention deficit disorders with hyperactivity and behavioural disorders.

Finally, the invention relates to the use of compounds of the formula I for the preparation of a medicament for the treatment of types of dementia of various origin, including vascular dementia, Lewy body dementia and dementia in Parkinson's disease.

The compounds of the formula I and processes for their preparation are disclosed in WO 01/07435. While being well tolerated, they exhibit, inter alia, effects on the central nervous system and have valuable pharmacological properties. The compounds have strong affinity to 5-HT_{2A} receptors and have 5-HT_{2A} receptor-antagonistic properties.

The compounds of the formula I are suitable both in veterinary and in human medicine for the treatment of functional disorders of the central nervous system and of inflammation. They can be used for the prophylaxis of and for combating the consequences of cerebral infarction (apoplexia cerebri), such as strokes (here, for example, trauma) and cerebral ischaemia and for the treatment of extrapyramidal-motory side effects of neuroleptics (for example dystonic syndrome, of muscle stiffness induced by neuroleptics, tremor (including substance-induced tremor forms) or extrapyramidal movement disorders), and of Parkinson's disease, including dopaminomimetic side effects of conventional Parkinson's medicaments, for the acute and symptomatic therapy of Alzheimer's disease and for the treatment of amyotrophic lateral sclerosis. They are likewise suitable as therapeutic agents for the treatment of brain trauma (for example after head injuries) or spinal cord trauma. However, they are particularly suitable as medicament active ingredients for anxiolytics, anti-depressives, antipsychotics, neuroleptics, antihypertonics and/or for positively influencing obsessive-compulsive disorder (OCD), including anancastic spectrum disorders (obsessive-compulsive spectrum disorders, OCSD), anxiety states, panic attacks, psychoses, schizophrenia, anorexia, delusional obsessions, agoraphobia, migraines, sleep disorders, tardive dyskinesia, learning disorders, age-dependent memory disorders, eating disorders, such as bulimia, drugs misuse (including disorders induced by substance misuse) and/or disorders of sexual function.

They are furthermore suitable for the treatment of endocrinic diseases, such as hyperprolactinaemia, furthermore in vasospasms, hypertension and gastrointestinal diseases.

They are furthermore suitable for the treatment of cardiovascular diseases and extrapyramidal symptoms, as described in WO 99/11641 on page 2, lines 24-30.

The compounds are in addition suitable for lowering the intraocular pressure and for the treatment of glaucoma.

The invention had the object of finding novel uses for medicaments prepared from the compounds of the formula I.

Surprisingly, it has been found that the compounds of the formula I are suitable for the treatment of obesity.

The efficacy of the compounds of the formula I for the treatment of obesity can be determined in vivo as follows (cf. Example B):

A certain dose or varying doses of the test compound is administered to one group of experimental animals over an extended period. The amount of feed consumed and the body weight of the experimental animals are recorded regularly. At the same time, the behaviour and general state of the animals are monitored in a manner known per se. Conclusions can be drawn on the suitability of the test compound for the treatment of obesity from the decrease in the body weight and the take-up of feed in the experimental period with an otherwise unchanged general state and in the absence of changes in behaviour.

The invention also relates to the use of compounds of the formula I for the preparation of a pharmaceutical preparation for the treatment of obesity comprising at least one medicament according to the invention and, if desired, excipients and/or adjuvants and, if desired, other active ingredients.

The medicaments can be converted into a suitable dosage form here together with at least one solid, liquid and/or semi-liquid excipient or adjuvant and, if desired, in combination with one or more further active ingredient(s).

For the treatment of obesity, the substances according to the invention are generally administered analogously to known preparations, preferably in doses of between about 0.1 and 500 mg, in particular between 5 and 300 mg per dosage unit. The daily dose is preferably between about 0.01 and 250 mg/kg, in particular between 0.02 and 100 mg/kg of body weight.

For the treatment of obesity, the substances according to the invention are preferably administered in doses of between about 1 and 500 mg, in particular between 5 and 100 mg, per dosage unit. The daily dose is preferably between about 0.02 and 10 mg/kg of body weight. However, the specific dose for each particular patient depends on a very wide variety of factors, for example on the efficacy of the specific compound employed, on the age, body weight, general state of health, sex, on the diet, on the time and method of administration, on the excretion rate, medicament combination and severity of the particular disorder to which the therapy applies. Oral administration is preferred.

It has furthermore been found that compounds of the formula I are suitable for the treatment of sub-types of anxiety states selected from a group consisting of social phobia, specific phobias, neophobia, post-traumatic stress disorders, acute stress anxiety, generalised anxiety disorders and bipolar disorders (mania).

A model of social phobia is the “social interaction test” as described by S. File, J. R. G. Hyde, J. Pharm. Pharmacol. 1977, 29: 735-738.

In this test, rats which do not know one another are placed in pairs in an open test box which is brightly illuminated (aversive condition), and it is recorded how often and for how long social contacts occur during a 5-minute test sitting.

A model of specific phobias is the shock sample test as described by D. Treit, M. A. Fundytus, Pharmacol. Biochem. Behav. 1988, 30: 1071-1075. In this test, individual rats are familiarised with an open, sawdust-filled box on 4 days for 30 minutes on each day. On the day of the test, a probe continuously carrying current is introduced into the box 2 cm above the base. The contacts with the probe are counted, and the attempts by the animals to cover the probe with sawdust are documented.

In a typical model of neophobia, foreign feed is made available to mice in a new environment after food has been denied for 18 hours [P. Soubrie et al., Psychopharmacologica, 1975, 45: 203-210], with the consumption of food being recorded.

Animal models of anxiety in connection with post-traumatic stress disorder work with the long-term changes in behaviour caused by the animals being exposed to a native stressor. The therapeutic effects of a substance which is effective for the acute treatment of anxiety in connection with posttraumatic stress are assessed in the model through the administration of the substance after confrontation with the stressor. The therapeutic effects of a substance which become evident during prophylactic treatment of anxiety in connection with post-traumatic stress are assessed in the model through the administration of the substance before confrontation with the stressor. Of the various behaviour test methods, the following is demonstrated the most frequently [R. E. Adamec and T. Shallow, Physiology Behavior, 1993, 54: 101-109; R. E. Adamec et al., Behav. Neurosci. 1997, 111: 435-449]. In general, a rat is exposed to a cat for five minutes, and the rat can be tested seven days later in a series of tests—the “hole board test”, in the “elevated-plus” labyrinth and in the “acoustic startle test”. The “hole board test” consists of a box (60 cm×60 cm) with four equally spaced holes; during a period of five minutes, it is counted how often the animal puts its head into a hole. The “elevated-plus” labyrinth consists of an X-shaped platform which is located above the base and has two “open” unprotected arms and two “closed” protected arms, the rats having free access to both types of arm. The rat is placed in the centre of the arms, and it is measured how often the animal ventures onto the open arms (risk assessment) and for how long it remains on the open and closed arms. In the “acoustic startle test”, the rat is placed in a Plexiglas cylinder and subjected to a series of 20 acoustic stimuli (“bursts”) in the form of white noise at 120 dB starting from a background noise of 60 dB; the latency time and the maximum startle amplitude are measured. In general, rats which have been exposed to a stressor, such as a cat, put their heads into the holes less often, have a lower risk assessment and a more pronounced fright reaction and spend less time on the open arms.

A typical model of acute stress anxiety is the “four plate test” as described by C. Aron et al., Neuropharmacology 1971, 10: 459-469.

The device consists of a small box whose base is composed of four metal plates. Each time the mouse moves from one plate to the next, it receives a short current pulse on the foot, thus reducing the investigative behaviour. The number of changes from one plate to the next which occur with punishment (i.e. the number of current pulses accepted by the animal) is recorded over a test period of 5 minutes. Normal mice make only few changes with punishment, i.e. they only accept a few current pulses on the foot, while (3-cyano-1H-indol-7-yl)[4-(4-fluorophenethyl)piperazin-1-yl]-methanone increases the number of changes with punishment.

A typical model of generalised anxiety disorders is the “light-dark choice test” as described by J. N. Crawly, Pharmacol. Biochem Behav. 1981, 15: 695-699.

The corresponding device for the selection of brightness or darkness consists of two boxes connected to one another, one box being darkened and the other being brightly illuminated. A mouse is placed in one of the boxes, and it is measured how much time the mouse spends in the illuminated box over a period of 5 minutes. Normal mice only go into the illuminated compartment rarely and spend almost all the time in the dark compartment. (3-Cyano-1H-indol-7-yl)[4-(4-fluorophenethyl)piperazin-1-yl]methanone increases the time spent by the mice in the illuminated compartment.

In a typical model of bipolar disorders (mania), a cycling activity (hyper- and hypolocomotory activity) is induced in rats by pharmacological means. The pharmacological stimuli used can be either ouabaine (El Mallakh, R. S. et al., Prog. Neuro-Psychopharmacol. Biol. Psychiatry, 1995; 19: 955-962) or amphetamine (Cappaliez, P. and Moore, E., Prog. Neuro-Psychopharmacol. Biol. Psychiatry, 1990; 14: 347-358). In another variant, mania symptoms are induced in rats by denying them sleep (Gessa, G. L. et al., Eur. Neuropsychopharmacol. 1995; 5 (Suppl.): 89-93).

The invention also relates to the use of compounds of the formula I for the preparation of a pharmaceutical preparation for the treatment of sub-types of anxiety states, comprising at least one medicament according to the invention and, if desired, excipients and/or adjuvants and, if desired, other active ingredients.

The medicaments here can be converted into a suitable dosage form together with at least one solid, liquid and/or semi-liquid excipient or adjuvant and, if desired, in combination with one or more further active ingredient(s).

For the treatment of sub-types of anxiety states, the substances according to the invention are generally administered analogously to known preparations, preferably in doses of between about 0.1 and 500 mg, in particular between 5 and 300 mg, per dosage unit. The daily dose is preferably between about 0.01 and 250 mg/kg, in particular between 0.02 and 100 mg/kg of body weight.

For the treatment of sub-types of anxiety states, the substances according to the invention are preferably administered in doses of between about 1 and 500 mg, in particular between 5 and 100 mg, per dosage unit. The daily dose is preferably between about 0.02 and 10 mg/kg of body weight. However, the specific dose for each particular patient depends on a very wide variety of factors, for example on the efficacy of the specific compound employed, on the age, body weight, general state of health, sex, on the diet, on the time and method of administration, on the excretion rate, medicament combination and severity of the particular disorder to which the therapy applies. Oral administration is preferred.

It has furthermore been found that the compounds of the formula are suitable for the treatment of sub-types of schizophrenia selected from a group consisting of schizotypical personality disorders, prevention of schizophrenia in first-degree relatives, treatment-resistant schizophrenia and psychosis in tardive dyskinesia.

A typical animal model of schizotypical personality disorders and the prevention of schizophrenia in first-degree relatives is restricted pre-pulse inhibition. Pre-pulse inhibition is a non-species-specific phenomenon in which the normal reflex reactions to discrete sensory events are reduced if slight prior stimulation has been carried out in advance; pre-pulse inhibition can be used to assess sensomotory gating processes. In patients with schizotypical personality disorders and their first-degree relatives (in whom there is a high risk of the development of schizotypical personality disorders), the normal function of pre-pulse inhibition is disturbed (Braff, D. et al., Psychophysiology, 1978; 15:339-343; Braff, D., Arch Gen Psychiatry, 1992; 49:206-215; Bolino, F. et al., Biol Psychiatry, 1994; 36:670-679). Recombinant in-bred mice having an inherently poor pre-pulse inhibition (crossing of the strains C57ML/6J and DBA/2J) represent a corresponding animal model (McCaughran, J. A. Jr. et al., Psychopharmacology 1997; 134:131-140; McCaughran, J. A. Jr. et al., Behav. Genetics, 1999; 29:21-30.). (3-Cyano-1H-indol-7-yl)[4-(4-fluorophenethyl)piperazin-1-yl]-methanone significantly reduces the deficits in pre-pulse inhibition in this recombinant mouse strain.

Animal models of treatment-resistant schizophrenia and psychosis in tardive dyskinesia are so-called stimulant-induced psychoses (Ellison, G., Brain Res. Rev. 1994; 137:193-197). Chronic misuse of ‘dopamine agonists’, such as, for example, amphetamine or cocaine, or antagonists of N-methyl-D-aspartate (NMDA), such as, for example, ketamine, phencyclidine and dizocilpine, causes a syndrome of behavioural effects which has many features in common with the symptom complex of schizophrenia, i.e. these substances induce schizophrenia-like symptoms in healthy people and accelerate psychotic reactions in stabilised schizophrenic patients (Luby, E. D. et al., Am. Med. Assoc. Arch. Neurol. Psychiatry, 1959; 119:61-67; Lahti, A. C. et al., Neuropsychopharmacology, 1995; 13:9-19; Malhotra, A. K. et al., Neuropsychopharmacology, 1996; 14:301-307; Adler, C. M. et al., Biol. Psychiatry 1998; 43:811-816). In rodents, these stimulants cause extreme hyperlocomotory activity (Schaefer, G. J. et al., Neuropharmacology 1984; 23:909-914; Millan, M. J. et al., Eur. J. Neurosci. 1999; 11:419-432; O'Neill, M. F. et al., Psychopharmacology, 1999, 145:237-250). In experiments with mice, (3-cyano-1H-indol-7-yl)[4-(4-fluorophenethyl)piperazin-1-yl]methanone significantly reduces hyperlocomotory activity induced by dizocilpine.

The invention also relates to the use of compounds of the formula I for the preparation of a pharmaceutical preparation for the treatment of sub-types of schizophrenia, comprising at least one medicament according to the invention and, if desired, excipients and/or adjuvants and, if desired, other active ingredients.

The medicaments here can be converted into a suitable dosage form together with at least one solid, liquid and/or semi-liquid excipient or adjuvant and, if desired, in combination with one or more further active ingredient(s).

For the treatment of sub-types of schizophrenia, the substances according to the invention are generally administered analogously to known preparations, preferably in doses of between about 0.1 and 500 mg, in particular between 5 and 300 mg, per dosage unit. The daily dose is preferably between about 0.01 and 250 mg/kg, in particular between 0.02 and 100 mg/kg of body weight.

For the treatment of sub-types of schizophrenia, the substances according to the invention are preferably administered in doses of between about 1 and 500 mg, in particular between 5 and 100 mg, per dosage unit. The daily dose is preferably between about 0.02 and 10 mg/kg of body weight. However, the specific dose for each particular patient depends on a very wide variety of factors, for example on the efficacy of the specific compound employed, on the age, body weight, general state of health, sex, on the diet, on the time and method of administration, on the excretion rate, medicament combination and severity of the particular disorder to which the therapy applies. Oral administration is preferred.

It has likewise been found that the compounds of the formula I, as defined above, are suitable for the treatment of diseases selected from a group consisting of aggression (including aggression disorders in youths and adults) and behavioural disorders in dementia (in particular in the elderly).

A typical animal model of aggression is the isolation-induced fighting behaviour in mice (Paivarinta, P., Pharmacol. Biochem. Behav., 1992; 42: 35-39; Haller, J. et al., Psychopharmacology, 1996; 126: 345-350). A mouse which has been kept isolated for a number of days exhibits an aggressive fighting behaviour if confronted with another mouse after the isolation time. The number of bites or bite attacks can be used as a measure of the aggression. (3-Cyano-1H-indol-7-yl)[4-(4-fluorophenethyl)piperazin-1-yl]methanone reduces the isolation-induced fighting behaviour in mice.

Since the same model can be used for aggression disorders in youths and adults, the results achieved in the aggression experiments suggest medical use of (3-cyano-1H-indol-7-yl)[4-(4-fluorophenethyl)piperazin-1-yl]-methanone in disorders of this type.

The animal models used for behavioural disorders in dementia are either learning-task models in rodents or monkeys for assessment of cognitive deficiencies in dementia or aggression models in rodents.

Of the learning models, the DMTS method (DMTS=delayed matching to sample) in monkeys (Buccafusco, J. J. et al., Psychopharmacology, 1995; 120: 256-266; Buccafusco, J. J. et al., Drug Dev. Res., 1996; 38: 196-203; Prendergast, M. A. et al., Pharmacol. Biochem. Behav., 1997; 56: 81-87) is potentially of interest since the various aspects of learning and memory can be measured in this method. An experiment begins with the illumination of a sample key provided with one of three coloured discs. The monkeys are taught to press the illuminated sample key in order to initiate the experiment. After the delay interval, the two keys available for selection illuminate, but not the sample key. One of the two keys available for selection is offered with the colour that the sample key had before the delay interval, while the other (wrong) key available for selection has one of the two other colours. If the monkey makes the correct assignment (i.e. if it presses the key available for selection which has the same colour as the stimulus key), the response is rewarded. The correct responses were selected in such a way that simple strategies, such as, for example, a position preference, changing between right/left or even changing between twice left and twice right, resulted in a hit rate at exactly the level of chance probability (50%). Finally, all stimulus equalisation measures were assigned to the delay duration. Monkeys have an individual ability to retain the same hit rate after delay times of various length, and the longest delay time selected for a certain monkey is that in which a hit rate somewhat greater than the chance probability (about 60% correct) is constantly possible. Under the conditions of these experiments, (3-cyano-1H-indol-7-yl)[4-(4-fluorophenethyl)piperazin-1-yl]methanone clearly improves the DMTS hit rate and a number of general aspects of working memory, including attention and recall from memory.

In a learning and memory model in rats, the T-labyrinth method is used, in which rats, after denial of food, have to learn to find a reward in the arm of the labyrinth which is opposite the arm in which the reward was offered in the previous experiment (Moran, P. et al., Brain Res. 1992; 569:156-158). (3-Cyano-1H-indol-7-yl)[4-(4-fluorophenethyl)piperazin-1-yl]methanone has memory-promoting effects in the learning process and facilitates better and faster learning with a shorter performance time.

The invention also relates to the use of compounds of the formula I for the preparation of a pharmaceutical preparation for the treatment of aggression (including aggression disorders in youths and adults) and behavioural disorders in dementia, comprising at least one medicament according to the invention and, if desired, excipients and/or adjuvants and, if desired, other active ingredients.

The medicaments here can be converted into a suitable dosage form together with at least one solid, liquid and/or semi-liquid excipient or adjuvant and, if desired, in combination with one or more further active ingredient(s).

For the treatment of aggression (including aggression disorders in youths and adults) and behavioural disorders in dementia, the substances according to the invention are generally administered analogously to known preparations, preferably in doses of between about 0.1 and 500 mg, in particular between 5 and 300 mg, per dosage unit. The daily dose is preferably between about 0.01 and 250 mg/kg, in particular between 0.02 and 100 mg/kg of body weight.

For the treatment of aggression (including aggression disorders in youths and adults) and behavioural disorders in dementia, the substances according to the invention are preferably administered in doses of between about 1 and 500 mg, in particular between 5 and 100 mg, per dosage unit. The daily dose is preferably between about 0.02 and 10 mg/kg of body weight. However, the specific dose for each particular patient depends on a very wide variety of factors, for example on the efficacy of the specific compound employed, on the age, body weight, general state of health, sex, on the diet, on the time and method of administration, on the excretion rate, medicament combination and severity of the particular disorder to which the therapy applies. Oral administration is preferred.

It has likewise been found that the compounds of the formula I are suitable for supplementary treatment in low-dose neuroleptic treatment.

For supplementary treatment in low-dose neuroleptic treatment, use can be made of the animal models of apomorphine-induced stereotypical behaviour patterns in mice (Protais, P. et al., Psychopharmacology, 1976; 50:1-6) or rats (Puech, A. J. et al., Eur. J. Pharmacol., 1978; 50:291-300). Antipsychosis medicaments cause strong inhibition of the apomorphine-induced stereotypical behaviour. If the simultaneous administration of the same ineffective substance increases the action of conventional neuroleptics, it is concluded that the substance is helpful for supplementary treatment in neuroleptic treatment with the aim of enabling lower doses of the neuroleptics to be used and thus of reducing their frequent side effects.

The invention also relates to the use of compounds of the formula I for the preparation of a pharmaceutical preparation for supplementary treatment in low-dose neuroleptic treatment, comprising at least one medicament according to the invention and, if desired, excipients and/or adjuvants and, if desired, other active ingredients.

The medicaments here can be converted into a suitable dosage form together with at least one solid, liquid and/or semi-liquid excipient or adjuvant and, if desired, in combination with one or more further active ingredient(s).

For supplementary treatment in low-dose neuroleptic treatment, the substances according to the invention are generally administered analogously to known preparations, preferably in doses of between about 0.1 and 500 mg, in particular between 5 and 300 mg, per dosage unit. The daily dose is preferably between about 0.01 and 250 mg/kg, in particular between 0.02 and 100 mg/kg of body weight.

For supplementary treatment in low-dose neuroleptic treatment, the substances according to the invention are preferably administered in doses of between about 1 and 500 mg, in particular between 5 and 100 mg, per dosage unit. The daily dose is preferably between about 0.02 and 10 mg/kg of body weight. However, the specific dose for each particular patient depends on a very wide variety of factors, for example on the efficacy of the specific compound employed, on the age, body weight, general state of health, sex, on the diet, on the time and method of administration, on the excretion rate, medicament combination and severity of the particular disorder to which the therapy applies. Oral administration is preferred.

It has furthermore been found that the compounds of the formula I are suitable for the treatment of types of dementia of various origin, including vascular dementia, Lewy body dementia and dementia in Parkinson's disease.

Typical experimental models of dementia are the passive avoidance test in rats [S. D. Glick and B. Zimmerberg, Behav. Biol., 1972, 7: 245-254; D. K. Rush, Behav. Neural Biol., 1988, 50: 255-274] and testing of memory functions by means of the Morris water maze test in relatively old rats [R. Morris, J. Neurosci. Methods, 1984, 11: 47-60; F. H. Gage et al.; Neurobiol. Aging. 1984, 5: 43-48].

The apparatus employed in the passive avoidance test consists of a track separated from a dark compartment by a small door. The amnesia-causing active ingredient scopolamine is administered to the animals before the acquisition experiment. The rat is placed at the beginning of the track opposite the dark compartment. The latency time before entry into the dark compartment is measured. As soon as the rat enters the dark compartment, the door is closed, and an electric shock is transmitted to the feet of the rat via a floor grid. 48 hours later, a retention experiment is carried out in accordance with the same pattern as the acquisition experiment (without scopolamine), and the latency time before entry into the dark compartment is again measured. Normal rats treated with scopolamine do not remember the electric shock from the acquisition experiment and, in the retention experiment, enter the dark compartment with a similar latency time.

The experimental set-up in the Morris water maze consists of a filled circular water tank with a diameter of 150 cm and an escape platform with a diameter of 15 cm which is mounted below the water surface 18 cm from the edge. The water is clouded so that the platform is invisible. If the rats are placed in the water tank, they swim around until they find the hidden platform by chance after a certain time (latency). This latency time before the platform is found serves as reference. After repeated training runs, the latency time before the platform is found shortens from day to day, i.e. the rats remember the position of the platform, they learn. Compared with young rats, relatively old rats learn more slowly over this period. They exhibit an impaired learning capacity. Effective medicaments against dementia and in particular Alzheimer's disease improve the learning capacity of relatively old rats.

The invention also relates to the use of compounds of the formula I for the preparation of a pharmaceutical preparation for the treatment of types of dementia of various origin, including vascular dementia, Lewy body dementia and dementia in Parkinson's disease, comprising at least one medicament according to the invention and, if desired, excipients and/or adjuvants and, if desired, other active ingredients.

The medicaments here can be converted into a suitable dosage form together with at least one solid, liquid and/or semi-liquid excipient or adjuvant and, if desired, in combination with one or more further active ingredient(s).

For the treatment of types of dementia of various origin, including vascular dementia, Lewy body dementia and dementia in Parkinson's disease, the substances according to the invention are generally administered analogously to known preparations, preferably in doses of between about 0.1 and 500 mg, in particular between 5 and 300 mg, per dosage unit. The daily dose is preferably between about 0.01 and 250 mg/kg, in particular between 0.02 and 100 mg/kg of body weight.

For the treatment of types of dementia of various origin, including vascular dementia, Lewy body dementia and dementia in Parkinson's disease, the substances according to the invention are preferably administered in doses of between about 1 and 500 mg, in particular between 5 and 100 mg, per dosage unit. The daily dose is preferably between about 0.02 and 10 mg/kg of body weight. However, the specific dose for each particular patient depends on a very wide variety of factors, for example on the efficacy of the specific compound employed, on the age, body weight, general state of health, sex, on the diet, on the time and method of administration, on the excretion rate, medicament combination and severity of the particular disorder to which the therapy applies. Oral administration is preferred.

Finally, it has been found that the compounds of the formula I are suitable for the treatment of Parkinson's disease (including idiopathic Parkinson's disease) and psychotic states which occur therein, such as, for example, visual and audible hallucinations, and dopaminomimetic side effects of conventional Parkinson's medicaments (for example all types of L-dopa- and dopamine agonist-induced dyskinesia, dystonia, motory fluctuations and psychotic states; such as, for example, visual and audible hallucinations) and/or of attention deficit disorders with hyperactivity and behavioural disorders.

A typical animal model of idiopathic Parkinson's disease and dopaminomimetic side effects of conventional Parkinson's medicaments is the Parkinson cynomolgus monkey as described by P. J. Blanchet et al., Exp.

Neurology 1998; 153: 214-222. Parkinson's symptoms are caused in monkeys by repeated injection of 1-methyl-4-phenyl-1,2,3,6-tetrahydropyridine (MPTP). The Parkinson's symptoms are assessed qualitatively on the “Laval University Disability Scale” (B. Gomez-Mancilla et al., 1993; Mov. Disord. 8: 144-150), with the following symptoms being measured: posture, mobility, climbing, gait, the retention of food, articulation, coat care, social interaction.

The animal model used for attention deficit hyperactivity disorders (ADHD) and behavioural disorders was the rat with spontaneous high pressure, since these rats are hyperactive and have a deficit in long-term attention in behaviour tasks (Sagvolden, T. et al., Physiol. Behav., 1993; 54: 1047-1055). The mutant coloboma mouse, which has phenotypical anomalies similar to ADHD, was recently introduced as an animal model (Wilson, M. C., Neurosci. Biobehav. Rev., 2000, 24: 51-57).

The invention also relates to the use of compounds of the formula I for the preparation of a pharmaceutical preparation for the treatment of Parkinson's disease (including idiopathic Parkinson's disease) and psychotic states which occur therein, of dopaminomimetic side effects of conventional Parkinson's medicaments and of attention deficit hyperactivity disorders and behavioural disorders, comprising at least one medicament according to the invention and, if desired, excipients and/or adjuvants and, if desired, other active ingredients.

The medicaments here can be converted into a suitable dosage form together with at least one solid, liquid and/or semi-liquid excipient or adjuvant and, if desired, in combination with one or more further active ingredient(s).

For the treatment of Parkinson's disease (including idiopathic Parkinson's disease) and psychotic states which occur therein, of dopaminomimetic side effects of conventional Parkinson's medicaments and of attention deficit hyperactivity disorders and behavioural disorders, the substances according to the invention are generally administered analogously to known preparations, preferably in doses of between about 0.1 and 500 mg, in particular between 5 and 300 mg, per dosage unit. The daily dose is preferably between about 0.01 and 250 mg/kg, in particular between 0.02 and 100 mg/kg of body weight.

For the treatment of Parkinson's disease (including idiopathic Parkinson's disease) and psychotic states which occur therein, of dopaminomimetic side effects of conventional Parkinson's medicaments and of attention deficit hyperactivity disorders and behavioural disorders, the substances according to the invention are preferably administered in doses of between about 1 and 500 mg, in particular between 5 and 100 mg, per dosage unit. The daily dose is preferably between about 0.02 and 10 mg/kg of body weight. However, the specific dose for each particular patient depends on a very wide variety of factors, for example on the efficacy of the specific compound employed, on the age, body weight, general state of health, sex, on the diet, on the time and method of administration, on the excretion rate, medicament combination and severity of the particular disorder to which the therapy applies. Oral administration is preferred.

The compounds according to the invention can also be employed together with other active ingredients in the treatment of the diseases mentioned.

A specific introduction to the synthesis of compounds of the formula I is given in WO 01/07435.

The pharmaceutical preparations can be employed as medicaments in human and veterinary medicine. Suitable excipients are organic or inorganic substances which are suitable for enteral (for example oral), parenteral or topical application and do not react with the novel compounds, for example water, vegetable oils, benzyl alcohols, polyethylene glycols, gelatine, carbohydrates, such as lactose or starch, magnesium stearate, talc or Vaseline. Suitable for enteral administration are, in particular, tablets, coated tablets, capsules, syrups, juices, drops or suppositories, suitable for parenteral application are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations.

The preparations indicated may be sterilised and/or comprise adjuvants, such as lubricants, preservatives, stabilisers and/or wetting agents, emulsifiers, salts for modifying the osmotic pressure, buffer substances, dyes, flavours and/or aroma substances. They can, if desired, also comprise one or more further active ingredients, for example one or more vitamins.

The examples below relate to pharmaceutical preparations:

### EXAMPLE A1

### Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised and sealed under sterile conditions. Each injection vial contains 5 mg of active ingredient.

### EXAMPLE A2

### Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE A3

### Solution

A solution is prepared from 1 g of an active ingredient of the formula I, 9.38 g of NaH₂PO₄×2H₂O, 28.48 g of NaH₂PO₄×12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE A4

### Ointment

500 mg of an active ingredient of the formula I are mixed with 99.5 g of Vaseline under aseptic conditions.

### EXAMPLE A5

### Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE A6

### Coated Tablets

Tablets are pressed analogously to Example E and subsequently coated in a conventional manner with a coating of sucrose, potato starch, talc, tragacanth and dye.

### EXAMPLE A7

### Capsules

2 kg of active ingredient of the formula I are introduced into hard gelatine capsules in a conventional manner in such a way that each capsule contains 20 mg of the active ingredient.

### EXAMPLE A8

### Ampoules

A solution of 1 kg of active ingredient of the formula I in 60 l of bidistilled water is transferred into ampoules, lyophilised under aseptic conditions and sealed under sterile conditions. Each ampoule contains 10 mg of active ingredient.

### EXAMPLE B

### Treatment of Dogs with (3-cyano-1H-indol-7-yl)[4-(4-fluoro-phenethyl)piperazin-1-yl]methanone, hydrochloride

The test substance in the form of hard gelatine capsules produced as described in Example B7 is administered once daily over a period of 11 days to a group of Beagle hounds about 8 months old which had been dewormed in advance. The amount of feed consumed is determined daily by re-weighing the unconsumed feed, the body weight before (day 0), during (day 5) and after (day 12) the feeding experiment. At the same time, the behaviour and general state of the animals are monitored over the entire experimental period. Drinking water in unlimited amount is available to the dogs.

As can be seen from the experimental results compiled in Table 1, the dogs increasingly restrict the consumption of feed during the experiment. This is associated with a significant reduction in body weight. The behaviour and general state of the dogs are not impaired in the experimental period by the administration of the test substance.

TABLE 1
Feeding experiment on Beagle hounds with (3-cyano-1H-indol-7-yl)
[4-(4-fluorophenethyl)piperazin-1-yl]-methanone, hydrochloride
(¹:body weight in kg,²:feed consumption in g).

| | Day | | | | | | | | | | | | |
|---|---|---|---|---|---|---|---|---|---|---|---|---|---|
| | 0 | 1 | 2 | 3 | 4 | 5 | 6 | 7 | 8 | 9 | 10 | 11 | 12 |
| Dose mg/kg | | 3 | 10 | 30 | 100 | 100 | 100 | 100 | 100 | 100 | 100 | 100 | |
| Dog 8910 (male)¹ | 8.6 | | | | | 8.6 | | | | | | | 7.8 |
| Dog 8936 (female)¹ | 7.5 | | | | | 7.3 | | | | | | | 6.2 |
| Dose mg/kg | | | | | | 50 | 50 | 50 | 50 | 50 | 50 | 50 | |
| Dog 1201 (male)¹ | 6.8 | | | | | 7.1 | | | | | | | 6.3 |
| Dog 2164 (female)¹ | 6.1 | | | | | 5.9 | | | | | | | 5.5 |
| Dose mg/kg | | 3 | 10 | 30 | 100 | 100 | 100 | 100 | 100 | 100 | 100 | 100 | |
| Dog 8910 (male)² | | 380 | 320 | 370 | 320 | 250 | 240 | 60 | 60 | 40 | 20 | 90 | |
| Dog 8936 (female)² | | 380 | 380 | 360 | 220 | 200 | 140 | 80 | 30 | 30 | 70 | 10 | |
| Dose mg/kg | | | | | | 50 | 50 | 50 | 50 | 50 | 50 | 50 | |
| Dog 1201 (male)² | | 440 | 260 | 400 | 230 | 340 | 220 | 160 | 170 | 130 | 80 | 40 | |
| Dog 2164 (female)² | | 310 | 270 | 280 | 280 | 340 | 180 | 260 | 150 | 140 | 130 | 60 | |

1. Use of compounds of the formula I 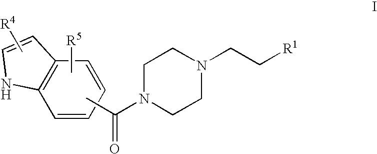
in which
R¹ is a phenyl radical or naphthyl radical, each of which is unsubstituted or substituted by R² and/or R³, or is Het¹,
R² and R³ are each, independently of one another, Hal, A, OA, OH or CN,
R⁴ and R⁵ are each, independently of one another, H, CN, acyl, Hal, A, OA or OH, CONH₂, CONHA or CONA₂,
R⁴ and R⁵ together are alternatively alkylene having 3-5 carbon atoms,
Het¹ is a monocyclic or bicyclic, unsaturated heterocyclic ring system which is unsubstituted or monosubstituted or disubstituted by Hal, A, OA or OH and which contains one, two or three identical or different heteroatoms, such as nitrogen, oxygen and sulfur,
A is alkyl having 1-6 carbon atoms,
Hal is F, Cl, Br or I,
and in which the indole ring may also be replaced by an isatin unit, and physiologically acceptable salts and solvates thereof, for the preparation of a medicament for the treatment of obesity.

2. Use of compounds of the sub-formula Ib of the formula I, in which
R¹ is phenyl, which is unsubstituted or monosubstituted by Hal,
and the radicals R⁴, R⁵ and Hal are as defined in claim 1, and physiologically acceptable salts and solvates thereof, for the preparation of a medicament for the treatment of obesity.

3. Use of compounds of the sub-formula Ii of the formula I, in which
R¹ is phenyl, naphthyl or Het¹, each of which is unsubstituted or monosubstituted by Hal,
R⁴ are each, independently of one another, H, Hal, CN, acyl, A or CONH₂,
R⁵ is H,
R⁴ and R⁵ together are alternatively alkylene having 3-5 carbon atoms,
Het¹ is thienyl or furyl, each of which is unsubstituted or monosubstituted or disubstituted by Hal or A,
and in which the indole ring may also be replaced by an satin unit, and physiologically acceptable salts and solvates thereof, for the preparation of a medicament for the treatment of obesity.

4. Use of compounds of the formula I selected from a group consisting of
(a) (3-cyano-1H-indol-7-yl)[4-(4-fluorophenethyl)piperazin-1-yl]-methanone,
(b) (3-aminocarbonyl-1H-indol-7-yl)[4-(4-fluorophenethyl)piperazin-1-yl]methanone and physiologically acceptable salts and solvates thereof, for the preparation of a medicament for the treatment of obesity.

5. Use of compounds of the formula I according to claim 1 and physiologically acceptable salts and solvates thereof, for the preparation of a medicament for the treatment of sub-types of anxiety states selected from a group consisting of social phobia, specific phobias, neophobia, post-traumatic stress disorders, acute stress anxiety, generalised anxiety disorders and bipolar disorders (mania).

6. Use of compounds of the sub-formula Ib of the formula I, in which
R¹ is phenyl, which is unsubstituted or monosubstituted by Hal,
and the radicals R⁴ and R⁵ are as defined in claim 1, and physiologically acceptable salts and solvates thereof, for the preparation of a medicament for the treatment of sub-types of anxiety states selected from a group consisting of social phobia, specific phobias, neophobia, post-traumatic stress disorders, acute stress anxiety, generalised anxiety disorders and bipolar disorders (mania).

7. Use of compounds of the sub-formula Ii of the formula I, in which
R¹ is phenyl, naphthyl or Het¹, each of which is unsubstituted or monosubstituted by Hal,
R⁴ are each, independently of one another, H, Hal, CN, acyl, A or CONH₂,
R⁵ is H,
R⁴ and R⁵ together are alternatively alkylene having 3-5 carbon atoms,
Het¹ is thienyl or furyl, each of which is unsubstituted or monosubstituted or disubstituted by Hal or A,
and in which the indole ring may also be replaced by an satin unit, and physiologically acceptable salts and solvates thereof for the preparation of a medicament for the treatment of sub-types of anxiety states selected from a group consisting of social phobia, specific phobias, neophobia, post-traumatic stress disorders, acute stress anxiety, generalised anxiety disorders and bipolar disorders (mania).

8. Use of compounds of the formula I selected from a group consisting of
(a) (3-cyano-1H-indol-7-yl)[4-(4-fluorophenethyl)piperazin-1-yl]-methanone,
(b) (3-aminocarbonyl-1H-indol-7-yl)[4-(4-fluorophenethyl)piperazin-1-yl]methanone
and physiologically acceptable salts and solvates thereof for the preparation of a medicament for the treatment of sub-types of anxiety states selected from a group consisting of social phobia, specific phobias, neophobia, post-traumatic stress disorders, acute stress anxiety, generalised anxiety disorders and bipolar disorders (mania).

9. Use of compounds of the formula I according to claim 1 and physiologically acceptable salts and solvates thereof for the preparation of a medicament for the treatment of sub-types of schizophrenia selected from a group consisting of schizotypical personality disorders, prevention of schizophrenia in first-degree relatives, treatment-resistant schizophrenia and psychosis in tardive dyskinesia.

10. Use of compounds of the sub-formula Ib of the formula I, in which
R¹ is phenyl which is unsubstituted or monosubstituted by Hal,
and the radicals R⁴ and R⁵ are as defined in claim 1, and physiologically acceptable salts and solvates thereof for the preparation of a medicament for the treatment of sub-types of schizophrenia selected from a group consisting of schizotypical personality disorders, prevention of schizophrenia in first-degree relatives, treatment-resistant schizophrenia and psychosis in tardive dyskinesia.

11. Use of compounds of the sub-formula Ii of the formula I, in which
R¹ is phenyl, naphthyl or Het¹, each of which is unsubstituted or monosubstituted by Hal,
R⁴ are each, independently of one another, H, Hal, CN, acyl, A or CONH₂,
R⁵ is H,
R⁴ and R⁵ together are alternatively alkylene having 3-5 carbon atoms,
Het¹ is thienyl or furyl, each of which is unsubstituted or monosubstituted or disubstituted by Hal or A,
and in which the indole ring may also be replaced by an satin unit, and physiologically acceptable salts and solvates thereof for the preparation of a medicament for the treatment of sub-types of schizophrenia selected from a group consisting of schizotypical personality disorders, prevention of schizophrenia in first-degree relatives, treatment-resistant schizophrenia and psychosis in tardive dyskinesia.

12. Use of compounds of the formula I selected from a group consisting of
(a) (3-cyano-1H-indol-7-yl)[4-(4-fluorophenethyl)piperazin-1-yl]methanone,
(b) (3-aminocarbonyl-1H-indol-7-yl)[4-(4-fluorophenethyl)piperazin-1-yl]methanone
and physiologically acceptable salts and solvates thereof for the preparation of a medicament for the treatment of sub-types of schizophrenia selected from a group consisting of schizotypical personality disorders, prevention of schizophrenia in first-degree relatives, treatment-resistant schizophrenia and psychosis in tardive dyskinesia.

13. Use of compounds of the formula I according to claim 1 and physiologically acceptable salts and solvates thereof for the preparation of a medicament for the treatment of diseases selected from a group consisting of aggression (including aggression disorders in youths and adults) and behavioural disorders in dementia.

14. Use of compounds of the sub-formula Ib of the formula I, in which
R¹ is phenyl which is unsubstituted or monosubstituted by Hal,
and the radicals R⁴ and R⁵ are as defined in claim 1, and physiologically acceptable salts and solvates thereof for the preparation of a medicament for the treatment of diseases selected from a group consisting of aggression (including aggression disorders in youths and adults) and behavioural disorders in dementia.

15. Use of compounds of the sub-formula Ii of the formula I, in which
R¹ is phenyl, naphthyl or Het¹, each of which is unsubstituted or monosubstituted by Hal,
R⁴ are each, independently of one another, H, Hal, CN, acyl, A or CONH₂,
R⁵ is H,
R⁴ and R⁵ together are alternatively alkylene having 3-5 carbon atoms,
Het¹ is thienyl or furyl, each of which is unsubstituted or monosubstituted or disubstituted by Hal or A,
and in which the indole ring may also be replaced by an isatin unit, and physiologically acceptable salts and solvates thereof for the treatment of diseases selected from a group consisting of aggression (including aggression disorders in youths and adults) and behavioural disorders in dementia.

16. Use of compounds of the formula I selected from a group consisting of
(a) (3-cyano-1H-indol-7-yl)[4-(4-fluorophenethyl)piperazin-1-yl]-methanone,
(b) (3-aminocarbonyl-1H-indol-7-yl)[4-(4-fluorophenethyl)piperazin-1-yl]methanone
and physiologically acceptable salts and solvates thereof for the preparation of a medicament for the treatment of diseases selected from a group consisting of aggression (including aggression disorders in youths and adults) and behavioural disorders in dementia.

17. Use of compounds of the formula I according to claim 1 and physiologically acceptable salts and solvates thereof for the preparation of a medicament for supplementary treatment in low-dose neuroleptic treatment.

18. Use of compounds of the sub-formula Ib of the formula I, in which
R¹ is phenyl which is unsubstituted or monosubstituted by Hal,
and the radicals R⁴ and R⁵ are as defined in claim 1, and physiologically acceptable salts and solvates thereof for the preparation of a medicament for supplementary treatment in low-dose neuroleptic treatment.

19. Use of compounds of the sub-formula Ii of the formula I, in which
R¹ is phenyl, naphthyl or Het¹, each of which is unsubstituted or monosubstituted by Hal,
R⁴ are each, independently of one another, H, Hal, CN, acyl, A or CONH₂,
R⁵ is H,
R⁴ and R⁵ together are alternatively alkylene having 3-5 carbon atoms,
Het¹ is thienyl or furyl, each of which is unsubstituted or monosubstituted or disubstituted by Hal or A,
and in which the indole ring may also be replaced by an satin unit, and physiologically acceptable salts and solvates thereof for the preparation of a medicament for supplementary treatment in low-dose neuroleptic treatment.

20. Use of compounds of the formula I selected from a group consisting of
(a) (3-cyano-1H-indol-7-yl)[4-(4-fluorophenethyl)piperazin-1-yl]-methanone,
(b) (3-aminocarbonyl-1H-indol-7-yl)[4-(4-fluorophenethyl)piperazin-1-yl]methanone
and physiologically acceptable salts and solvates thereof for supplementary treatment in low-dose neuroleptic treatment.
